(19) United States
(12) Patent Application Publication
(10) Pub. No.: US 2003/0157058 A1
(43) Pub. Date: Aug. 21, 2003

(54) Troponin anti-metastatic therapeutic and method of use

(76) Inventor: Marc E. Lanser, Dover, MA (US)

Correspondence Address: BROWN, RUDNICK, BERLACK & ISRAELS, LLP., BOX IP, 18TH FLOOR, ONE FINANCIAL CENTER, BOSTON, MA, 02111, US

(21) Appl. No.: 10/337,461

(22) Filed: Jan. 7, 2003

Related U.S. Application Data
(60) Provisional application No. 60/347,152, filed on Jan. 9, 2002.

(51) Int. Cl. A61K 38/19

(52) U.S. Cl. 424/85.1

(57) ABSTRACT

This invention comprises a method of inhibiting cancer metastases in a subject by the step of administering to said subject a therapeutically effective amount of a composition selected from the group consisting of Troponin, TnI subunits, analogs, derivatives, fragments or homologs.

### RELATED APPLICATION

This application claims the benefit from U.S. Provisional application serial No. 60/347,152 filed Jan. 9, 2002 which is incorporated herein by reference in its entirety.

### BACKGROUND OF THE INVENTION

Troponin I (TnI) is a 181 amino acid, 21 kDa protein, one of the three polypeptide components (along with Troponin C and Troponin T) which comprise the muscle troponin complex. Heterotrimeric troponin binds actin thin filaments in muscle and is intimately involved with Ca⁺⁺-dependent muscle contraction. Analysis of the troponin complex reveals several distinct functional motifs for the TnI subunit: amino acids 1-47 of TnI are involved with binding to Troponin C, amino acids 96-116 comprise the “active site” involved with actomyosin ATPase activity, and the C-terminal amino acids 166-182 are involved with binding TnI to the actin filaments. Troponin is a therapeutically effective anti-angiogenic composition.

Angiogenesis, the process of new blood vessel development and formation. Pathological angiogenesis characterizes solid tumor growth which sustains progression cancer is a subject.

### SUMMARY OF THE INVENTION

In particular embodiments this invention comprises an anti-metastatic therapeutic and method of its use. Troponin is therapeutically effective in suppression of malignant metastases. Particular reference is made to suppression of cancerous lung metastases. In some embodiments suppression is greater than about 90%. Melanoma metastases are significantly inhibited by constant infusion of low dose Troponin I. In some embodiments, Troponin I is administered to maintain a therapeutically effective blood level for extended periods with reference to about 1 day or longer, about 10 days or longer, and about 30 days and including chronic administration indefinitely. In certain applications, Troponin I, is administered by constant infusion in low doses. This therapeutic regimen dramatically suppressed growth of melanoma metastases in the lung. These results, illuminate a dosage protocol applicable to human therapeutic uses of Troponin. Troponin has efficacy as a clinically effective anti-angiogenic therapeutic for the prevention of tumor metastases in humans, with specific reference to high-risk cancer patients.

### DETAILED DESCRIPTION OF THE INVENTION

This invention will be better understood with reference to the following definitions:

A. Troponin I (TnI) shall mean a 181 amino acid, 21 kDa protein, best known as one of the three polypeptide components (along with Troponin C and Troponin T) which comprise the muscle troponin complex. Heterotrimeric troponin binds actin thin filaments in muscle and is intimately involved with Ca⁺⁺-dependent muscle contraction. Without being bound by specific structural aspects, it is believed troponin complex exhibits several distinct functional motifs for the TnI subunit. These motifs include: amino acids 1-47 of TnI apparently involved with binding to Troponin C, amino acids 96-116 apparently comprising the “active site” involved with actomyosin ATPase activity, and the C-terminal amino acids 166-182 apparently involved with binding TnI to the actin filaments. This is also termed Cardiac troponin I (cTnI).

The Troponin C subunit of troponin is believed to be the Ca2+ binding subunit of troponin. It is further believed to contain two homologous domains and four divalent cation binding sites. Two structural sites in the C-terminal domain of troponin C bind either Ca2+ or Mg2+, and two regulatory sites in the N-terminal domain are specific for Ca2+.

Therapeutically effective analogs of troponin subunits C, I, or T and analogs of their fragments are also contemplated within the practice of this invention. Troponin subunit fragments can be made by altering troponin sequences by substitutions, additions or deletions that provide for functionally equivalent molecules. These include, but are not limited to, troponin subunits, fragments, or analogs containing, as a primary amino acid sequence, all or part of the amino acid sequence of a troponin subunit including altered sequences in which functionally equivalent amino acid residues are substituted for residues within the sequence resulting in a silent change. For example, one or more amino acid residues within the sequence can be substituted by another amino acid of a similar polarity which acts as a functional equivalent, resulting in a silent alteration. Substitutes for an amino acid within the sequence may be selected from other members of the class to which the amino acid belongs. For example, the nonpolar (hydrophobic) amino acids include alanine, leucine, isoleucine, valine, proline, phenylalanine, tryptophan and methionine. The polar neutral amino acids include glycine, serine, threonine, cysteine, tyrosine, asparagine, and glutamine. The positively charged (basic) amino acids include arginine, lysine and histidine. The negatively charged (acidic) amino acids include aspartic acid and glutamic acid.

B. Low dose shall be understood to mean about 5%, and particular less than about 2% and more particularly about 1% or less of the quantity of Troponin currently being used in human clinical studies as compared with other anti-angiogenic proteins. In the practice of this invention, low doses provided a therapeutically effective dose. In humans, a dosage of about 1 to about 5 mg/kg/day is a low dose.

Therapeutically effective amount as to a drug dosage, shall be broadly understood to mean that dosage that provides the specific pharmacological response for which the drug is administered in a significant number of subjects in need of such treatment. Reference to “specific pharmacological response for which the drug is administered in a significant number of subjects in need of such treatment” is a recognition that a “therapeutically effective amount,” administered to a particular subject in a particular instance will reduce the number of metastases as anticipated based on a statistical determination. Therapeutically effective in such context offers a reduction of metastases, which while not in itself specifically curative, is palliative and useful in prolonging a subject's life and/or prolonging the stage at which a subject is treatable with an expectation of clinical improvement or survival.

Reference is made to “Tumstatin, an endothelial cell-specific inhibitor of protein synthesis,” Maeshima et al., Science Jan. 4, 2002;295(5552):140-3; “Endostatin reduces vascularization, blood flow, and growth in a rat gliosarcoma,” Sorensen, Neuro-oncol 2002 January;4(1):1-8; “Thrombospondin-1-mediated metastasis suppression by the primary tumor in human melanoma xenografts,” Rofstad, J Invest Dermatol 2001 November; 117(5):1042-9; “Experience with anti-angiogenic therapy of giant cell granuloma of the facial bones.” Collins A. Ann R Australas Coll Dent Surg 2000 October;15:170-5; “Potent anti-metastatic activity of combretastatin-A4,” Griggs, Int. J Oncol 2001 October;19(4):821-5; and “Improved survival in tumor-bearing SCID mice treated with interferon-gamma-inducible protein 10 (IP-10/CXCL10),” Arenberg, Cancer Immunol Immunother 2001 December;50(10):533-8, the teachings of which are incorporated herein by reference.

C. Metastases shall be broadly understood to include both cancer that started from cancer cells from another part of the body, the “primary site” (for example: cancer that starts in the breast can spread to the lymph nodes and then be spread throughout the body), as well as cancer starting at one site in a particular part of the body appearing at a separate site in the same part of the body (for example: a second lung tumor of a given type appearing after a first lung tumor of that type begins).

D. Suppression or inhibition of metastases by the method of this invention shall mean Troponin I or TnI subunits, analogs, derivatives, fragments or homologs is therapeutically effective in suppression of malignant metastases. Reduction of the number of metastases is calculated in comparison to the number predicted based on a statistical determination of published or clinically established results of populations with similar cancers at similar stages. Particular reference is made to suppression of cancerous lung metastases. In some embodiments suppression is greater than about 90%. Melanoma metastases are significantly inhibited by constant infusion of low dose Troponin I. In some embodiments, Troponin or analogs or fragments thereof is administered to maintain a therapeutically effective blood level for extended periods with reference to about 1 day or longer. Particular reference is made to a therapeutically effective dosage level is chronically maintained for at least about 5 days, at least about 10 days, at least about 20 days, and at least about 30 days or longer with particular reference to indefinite continuation, i.e. for the life of the subject.

In certain applications, Troponin, was administered by constant infusion in low doses to mice. This therapeutic regimen dramatically suppressed growth of melanoma metastases in the lung. These results, illuminate a dosage protocol applicable to human therapeutic uses of Troponin. Troponin has efficacy as a clinically effective anti-angiogenic therapeutic for the prevention of tumor metastases in humans, with specific reference to high-risk cancer patients.

TABLE 1
MALIGNANCIES AND RELATED DISORDERS
Solid tumors

| | | |
|---|---|---|
| sarcomas and carcinomas | breast cancer | Wilms' tumor |
| fibrosarcoma | ovarian cancer | cervical cancer |
| myxosarcoma | prostate cancer | testicular tumor |
| liposarcoma | squamous cell | lung carcinoma |
| | carcinoma | |
| chondrosarcoma | basal cell carcinoma | small cell lung |
| | | carcinoma |
| osteogenic sarcoma | adenocarcinoma | bladder carcinoma |
| chordoma | sweat gland carcinoma | glioma astrocytoma |
| angiosarcoma | sebaceous gland | medulloblastoma |
| | carcinoma | |
| endotheliosarcoma | papillary carcinoma | craniopharyngioma |
| lymphangiosarcoma | papillary | ependymoma |
| | adenocarcinomas | |
| llymphangioendothelios | cystadenocarcinoma | Kaposi's sarcoma |
| arcoma | medullary carcinoma | pinealoma |
| synovioma | bronchogenic | hemangioblastoma |
| | Carcinoma | |
| mesothelioma | renal cell carcinoma | acoustic neuroma |
| Ewing's tumor | hepatoma | oligodendroglioma |
| Leiomyosarcoma | bile duct carcinoma | menagioma melanoma |
| rhabdommyosarcoma | choriocarcinoma | neuroblastoma |
| colon carcinoma | seminoma | retinoblastoma |
| pancreatic cancer | embryonal carcinoma | |

Without being bound by any particular theory, it is believed that Troponin, Troponin subunits, analogs, derivatives, fragments or homologs inhibit metastases in a variety of cancers.

Publications: The following publications, all of which are incorporated herein by reference are presented by number. The numbering is offered as a matter of convenience.

1. Farah, C. S. and Reinach, F. C. (1995). The troponin complex and regulation of muscle contraction. FASEB J. 9, 755-767.

2. Coudrey, L. (1998). The troponins. Archives of Int. Med. 158(11), 1173-1180.

3. Filatov, V. L., Katrukha, A. G., Bulargina, T. V., and Gusev, N. B. (1999). Troponin: structure, properties, and mechanism of functioning. Biochemistry (Mosc.) 64, 969-985.

4. Greaser, M. L. and Gergely, J. (1971). Reconstitution of troponin activity from three protein components. J Biol Chem. 246, 4220-4233.

5. Yates, L. D. and Greaser, M. L. (1983). Troponin subunit stoichiometry and content in rabbit skeletal muscle and myofibrils. J Biol Chem. 258, 5770-5774.

6. Farah, C. S., Miyamoto, C. A., Ramos, C. H. I., Silva, A. C. R., Quaggio, R. B., Fujimori, K., Smillie, L. B., and Reinach, F. C. (1994). Structural and regulatory functions of the NH₂- and COOH-terminal regions of skeletal muscle troponin I. J Biol Chem. 269, 5230-5240.

7. Ramos, C. H. (1999). Mapping subdomains in the C-terminal region of troponin I involved in its binding to troponin C and to thin filament. J Biol Chem. 274, 18189-18195.

8. Tao, T., Gong, B. J., Grabarek, Z., and Gergely, J. (1999). Conformational changes induced in troponin I by interaction with troponin T and actin/tropomyosin. Biochim Biophys Acta. 1450, 423-433.

9. Hernandez, G., Blumenthal, D. K., Kennedy, M. A., Unkefer, C. J., and Trewhella, J. (1999). Troponin I inhibitory peptide (96-115) has an extended conformation when bound to skeletal muscle troponin C. Biochemistry. 38, 6911-6917.

10. Moses, M. A., Wiederschain, D., Wu, I., Fernandez, C. A., Ghazizadeh, V., Lane, W. S., Flynn, E., Sytkowski, A., Tao, T., and Langer, R. (1999). Troponin I is present in human cartilage and inhibits angiogenesis. Proc. Natl. Acad. Sci. USA. 96, 2645-2650.

11. Zhu, L., Perez-Alvarado, G., and Wade, R. (1994). Sequencing of a cDNA encoding the human fast-twitch skeletal muscle isoform of troponin I. Biochim Biophys Acta. 1217, 338-340.

12. Connolly, D. T., Knight, M. B., Harakas, N. K., Wittwer, A. J., Feder, J. (1986). Determination of the number of endothelial cells in culture using an acid phosphatase assay. Anal. Biochem. 152, 136-140.

13. Hansen, M. B., Nielsen, S. E., and Berg, K. (1989). Re-examination and further development of a precise and rapid dye method for measuring cell growth/cell kill. J Immunol. Meth. 119,203-210.

14. Feldman, L., Cohen, C. M., Riordan, M. A., and Dainiak, N. (1987). Purification of a membrane-derived erythroid growth factor. Proc. Natl. Acad. Sci. USA. 84, 6775-6779.

15. Lowry, O. H., Rosebrough, N. J., Farr, A. L., Randall, R. J. (1951). Protein measurement with the Folin phenol reagent. J Biol. Chem. 193, 265-275.

16. Laemmli, U. K. (1970). Cleavage of structural proteins during the assembly of the head of bacteriaphage T4 . Nature. 227, 680-685.

17. Towbin, H. Staehlin, T., and Gordon, J. (1979). Electrophoretic transfer of proteins from polyacrylamide gels to nitrocellulose sheets: procedures and applications. Proc. Natl. Acad. Sci. USA 76:4350-4354.

18. Wiederschain D G et al., Patent Number: WO0054770, Publication date: Sep. 21, 2000.

19. U.S. Pat. No. 5,837,680 Moses et al., Pharmaceutical compositions comprising troponin subunits, fragments and analogs thereof and methods of their use to inhibit angiogenesis.

This invention will be further understood with reference to the following examples.

### EXAMPLE 1

Troponin I was administered to mice beginning 24 hours after intravenous introduction of B16 melanoma cells into the animals. These melanoma cells localize in the lung within several hours after injection of the melanoma cells. Without being bound by any particular theory, it is believed that these melanoma cells require the establishment of a vascular blood supply in order to grow to a size that is visible to the naked eye. After melanoma introduction, the mice were treated with a constant intravenous infusion of Troponin at a cumulative dose of either 1 or 5 mg/kg/day. Animals were sacrificed after 24 days of treatment. Animals receiving 1 mg/kg/day of Troponin had an approximate 65% decrease in the number of lung metastases compared to control, and animals receiving 5 mg/kg/day had a greater than 90% decrease in the number of metastases compared to control. Maintained intravenous infusion of Troponin at very low doses results in a dramatic reduction in metastatic tumor growth.

### EXAMPLE 2

A 56 year old human male presents with malignant melanoma localized in the lung. This patient is treated with a constant intravenous infusion of Troponin at a cumulative dose of 1 mg/kg/day every other day for 90 days. Less than about 35% of the anticipated number of lung metastases are detected as compared with a statistical evaluation of published results of patients at a similar stage of melanoma.

### EXAMPLE 3

A 60 year old human female presents with malignant melanoma localized in the lung. This patient is treated with a constant intravenous infusion of Troponin at a cumulative dose of 3 mg/kg/day every day for 25 days. At 25 days after first dose, less than about 50% of the anticipated number of lung metastases are detected as compared with a statistical evaluation of published results of patients at a similar stage of melanoma.

### EXAMPLE 4

A 44 year old human male presents with malignant melanoma localized in the lung. This patient is treated with a constant intravenous infusion of Troponin at a cumulative dose of 5 mg/kg/day. Every day for 90 days. At 90 days after first dose, less than about 80% of the anticipated number of lung metastases are detected as compared with a statistical evaluation of published results of patients at a similar stage of melanoma.

The compositions and methods of this invention possess valuable pharmacological properties. They restrict cell proliferation and reduce the establishment and/or occurrence of cancer metastases with particular reference to malignant melanoma, a further reference to malignant melanoma of the lung.

Thus, these compositions can be used in the therapeutic treatment of a number of forms of cancers, with particular reference to those noted in Table I. Administration is contemplated to include chronic, acute or intermittent regimens.

The compositions of this invention are generally administered to animals, including but not limited to mammals including livestock, household pets, humans, cattle, cats, dogs, poultry, etc. Particular reference to the methods and compositions is made for the treatment of human subjects in need of such treatment such as those suffering from cancer or other cell proliferative conditions.

The pharmacologically active compositions of this invention can be processed in accordance with conventional methods of Galenic pharmacy to produce medicinal agents for administration to patients, e.g., mammals including humans.

The compositions of this invention can be employed in admixture with conventional excipients, i.e., pharmaceutically acceptable organic or inorganic carrier substances suitable for parenteral, enteral (e.g., oral or inhalation) or topical application which do not deleteriously react with the active compositions. Inhalation is particularly useful in treating or dosing lung tissue, including free Troponin or liposomal Troponin in aerosol form. Reference is made to U.S. Pat. No. 5,049,388 (Knight) regarding liposomal aerosol, the teachings of which are incorporated by reference. Suitable pharmaceutically acceptable carriers include but are not limited to water, salt solutions, alcohols, gum arabic, vegetable oils, benzyl alcohols, polyethylene glycols, gelatine, carbohydrates such as lactose, amylose or starch, magnesium stearate, talc, silicic acid, viscous paraffin, perfume oil, fatty acid esters, hydroxy methylcellulose, polyvinyl pyrrolidone, etc. The pharmaceutical preparations can be sterilized and if desired mixed with auxiliary agents, e.g., lubricants, preservatives, stabilizers, wetting agents, emulsifiers, salts for influencing osmotic pressure, buffers, coloring, flavoring and/or aromatic substances and the like which do not deleteriously react with the active compositions. They can also be combined where desired with other active agents, e.g., vitamins.

In some embodiments of the present invention, dosage forms include instructions for the use of such compositions. In some embodiments administration is in conjunction with other antineoplastic drugs and or in conjunction with radiation therapy.

For parenteral application, particularly suitable are injectable, sterile solutions, preferably oily or aqueous solutions, as well as suspensions, emulsions, or implants, including suppositories. Ampules are convenient unit dosages.

Also for parenteral application, particularly suitable are tablets, dragees, liquids, drops, suppositories, or capsules. A syrup, elixir, or the like can be used wherein a sweetened vehicle is employed.

Sustained or directed release compositions can be formulated, e.g., liposomes or those wherein the active component is protected with differentially degradable coatings, e.g., by microencapsulation, multiple coatings, etc. It is also possible to freeze-dry the new compositions and use the lyophilizates obtained, for example, for the preparation of products for injection.

Generally, the compositions of this invention are dispensed in unit dosage form comprising 0.1 to 500 mg or more in a pharmaceutically acceptable carrier per unit dosage.

It will be appreciated that the actual preferred amounts of active compositions in a specific case will vary according to the specific compositions being utilized, the particular compositions formulated, the mode of application, and the particular situs and organism being treated. Dosages for a given host can be determined using conventional considerations, e.g., by customary comparison of the differential activities of the subject compositions and of a known agent, e.g., by means of an appropriate, conventional pharmacological protocol.

1. A method of inhibiting cancer metastases in a subject by the step of administering to said subject a therapeutically effective amount of a composition selected from the group consisting of TnI, TnI subunits, analogs, derivatives, fragments or homologs.

2. The method of claim 1 wherein the cancer is malignant melanoma.

3. The method of claim 1 wherein the primary site of the malignant melanoma is the lung.

4. The method of claim 1 where administration comprises at least about 1 mg/kg/day.

5. The method of claim 4 wherein said administration is continuous for at least about 10 days.

6. The method of claim 4 wherein said administration is every other day for at least about 10 days.

7. The method of claim 4 wherein a therapeutically effective dosage level is indefinitely maintained.

8. The method of claim 1 where administration comprises at least about 5 mg/kg/day.

9. The method of claim 8 wherein said administration is continuous for at least about 10 days.

10. The method of claim 8 wherein said administration is every other day for at least about 10 days.

11. The method of claim 8 wherein a therapeutically effective dosage level is indefinitely maintained.

12. A method of inhibiting cancer metastases by at least about 35% of the anticipated number of metastases in a subject by the step of administering to said subject a therapeutically effective amount of a composition selected from the group consisting of TnI, TnI subunits, analogs, derivatives, fragments or homologs.

13. The method of claim 12 wherein said inhibition is at least about 50%.

14. The method of claim 12 wherein said inhibition is at least about 80%.

15. The method of claim 12 wherein said inhibition is at least about 90%.
